(19) United States
(12) Patent Application Publication
(10) Pub. No.: US 2014/0257402 A1
(43) Pub. Date: Sep. 11, 2014

(54) SURGICAL SYSTEM FOR POSITIONING A PATIENT AND MARKING LOCATIONS FOR A SURGICAL PROCEDURE

(71) Applicant: THE CLEVELAND CLINIC FOUNDATION, Cleveland, OH (US)

(72) Inventor: Wael K. Barsoum, Bay Village, OH (US)

(73) Assignee: THE CLEVELAND CLINIC FOUNDATION, Cleveland, OH (US)

(21) Appl. No.: 14/198,627

(22) Filed: Mar. 6, 2014

Related U.S. Application Data
(60) Provisional application No. 61/774,623, filed on Mar. 8, 2013.

(51) Int. Cl. A61G 13/10; A61B 17/70

(52) U.S. Cl. CPC A61G 13/10; A61B 17/7076; A61B 2017/320052
USPC 606/279; 5/652; 5/655.9; 5/621; 606/96

(57) ABSTRACT

A device for positioning a patient on a surgical table for a surgical procedure includes a main body with a footprint-receiving portion configured to accept and permanently maintain a footprint of at least a portion of a contact tissue of a patient's body. The footprint imposes a desired position of the contact tissue for the surgical procedure.

### RELATED APPLICATION

This application claims priority from U.S. Provisional Application No. 61/774,623, filed 8 Mar. 2013, the subject matter of which is incorporated herein by reference in its entirety.

### TECHNICAL FIELD

The present invention relates generally to a surgical system and, more particularly, to a surgical system for positioning a patient on an operating table for a surgical procedure.

### BACKGROUND

The spine is a complex anatomical structure that provides protection for the spinal cord and support for a patient. The spine includes both hard tissue portions (e.g., vertebral bodies, pedicles, and processes) and soft tissue portions (e.g., intervertebral disks, nerves, ligaments, and connecting tissue). Due to the varying forces and pressures exerted on the spine, a surgical procedure may be helpful to restore structural stability of an injured spine. Often, screws will be inserted into one or more pedicles to fixate the spine.

Spinal surgical procedures are often used to treat the patient and restore at least a portion of spinal stability. Due to the complex nature of the spine and for the health of the patient, minimally invasive spinal procedures may be desirable in some patients. However, it may be difficult for a surgeon to pinpoint or “eyeball” a specific portion of the spine that needs a surgical procedure. To resolve this problem, surgical positioning assemblies have been developed for use with operating tables to help position a patient for a spinal procedure. These surgical positioning assemblies mirror a silhouette or a contoured imprint of a desired position of the patient for the surgical procedure. A surface of the surgical positioning assemblies may accept and temporarily maintain the silhouette or contoured imprint of the desired position of the patient. The surfaces of the existing surgical positioning assemblies may be configured to deform as a patient lies on the surface. The composition of the surface allows the surface to be capable of temporarily accepting a silhouette or a contoured imprint of the position of the patient as the patient lies on the surgical positioning system. Additionally, a patient-specific instrument (hereafter, “PSI”) that corresponds to the portion of that particular patient's native tissue, (e.g., spine) may be provided. When present, the PSI may be placed on the skin surface that overlies the portion of the native tissue (e.g., one or more pedicles) to be operated on.

### SUMMARY

In an embodiment of the present invention, a device for positioning a patient on a surgical table for a surgical procedure is disclosed. The device includes a main body with a footprint-receiving portion that is configured to accept and permanently maintain a footprint of at least a portion of a contact tissue of a patient's body. The footprint imposes a desired position of the contact tissue for the surgical procedure.

In an embodiment of the present invention, a system for positioning a patient on a surgical table for a surgical procedure and implementing a preoperative surgical plan is disclosed. The system includes a surgical device that has a main body with a footprint-receiving portion. The footprint-receiving portion is configured to accept and permanently maintain a footprint of at least a portion of a contact tissue. The footprint imposes a desired position of the contact tissue for a surgical procedure. A patient-specific instrument is configured to reflect characteristics of at least a portion of a native tissue. The patient-specific instrument is manufactured responsive to images taken of the portion of native tissue while the patient is positioned on the surgical device.

In an embodiment of the present invention, a method for preparing and implementing a preoperative surgical plan is disclosed. A patient is positioned on a surgical device before a surgical procedure. The surgical device has a footprint-receiving portion that is configured to accept and permanently maintain a footprint of at least a portion of a contact tissue as arranged in a desired position for the surgical procedure. The footprint-receiving portion is configured to impose a position of the contact tissue when the contact tissue contacts at least a portion of the main body. The patient is returned to the position imposed in the footprint-receiving portion of the main body. A patient-specific instrument is aligned with at least a portion of a native tissue. At least one surgical aperture is formed into the native tissue guided by at least one opening of the patient-specific instrument. At least one location is marked within the at least one surgical aperture with a marking structure.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 is an exploded view showing a surgical system according to an embodiment of the present invention;

FIG. 2 is a perspective view showing a first component of the system of FIG. 1;

FIG. 3 is a perspective view showing the first component of FIG. 1;

FIG. 4A is a top view showing a second component of the system of FIG. 1;

FIG. 4B is a perspective view showing the second component of FIG. 1;

FIG. 5 is a bottom view showing the second component of FIG. 1;

FIG. 6 is a perspective view showing the second component of FIG. 1; and

FIGS. 7-12 illustrate an example sequence of operation of the embodiment of FIG. 1.

### DETAILED DESCRIPTION

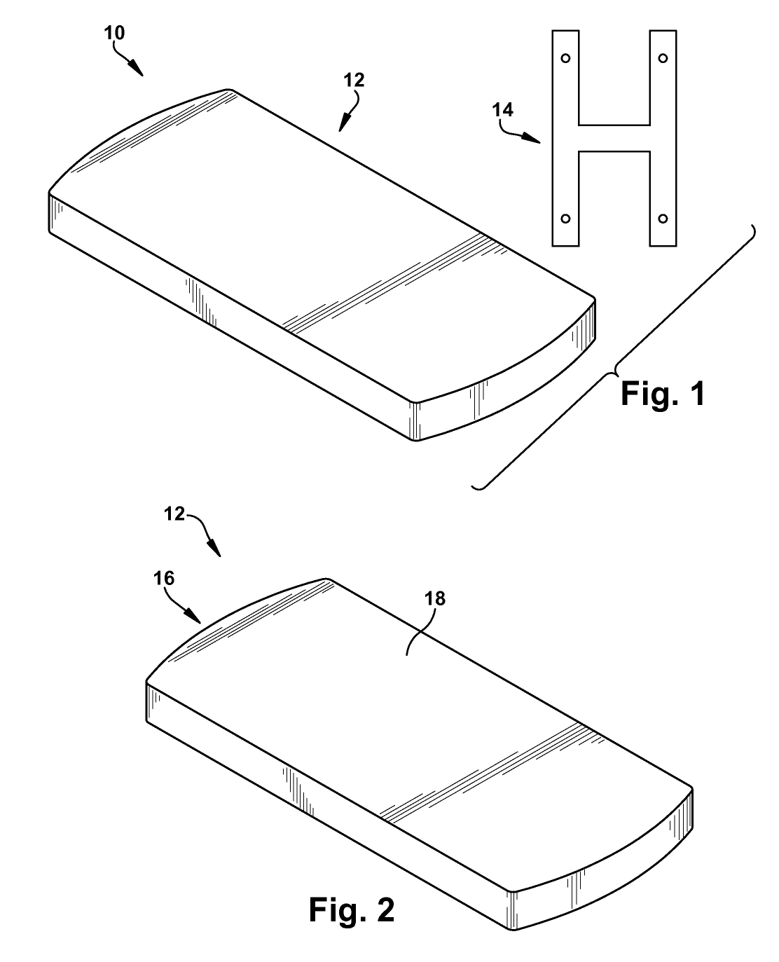

Although the present invention is described below primarily in terms of a spinal procedure, it will be appreciated that the system may be used during any surgical procedure (e.g., procedures for a shoulder, knee, neck, hip, ankle, phalangeal, metacarpal, metatarsal, a fracture site of long bones, muscles, tendons, ligaments, cartilage, or any other patient tissue). According to an aspect of the present invention, FIG. 1 illustrates a system 10 for positioning a patient for a surgical procedure and marking locations on a portion of a native tissue of the patient. As used herein, the term “native tissue” (and variants thereof) refers to a portion of the patient's body, including a portion of a skin surface and a portion of an underlying subcutaneous tissue that is of interest in its condition at the time of surgical preparation, having any included natural or artificial structures of interest, whether congenital or acquired. As used herein, the term “subcutaneous tissue” (and variants thereof) refers to a portion of native tissue located beneath a skin surface. Specifically, the term “subcutaneous tissue” is used in the below example to refer to a portion of the spine, and, more specifically, the term “subcutaneous tissue” will be used herein to reference one or more pedicles of the patient's spine. As used herein, the term “skin surface” (and variants thereof) refers to a layer of skin tissue that overlies the subcutaneous tissue. However, it will be appreciated that the system 10 may be used in connection with any portion of a subcutaneous tissue and a corresponding skin surface of the patient (e.g., arm, shoulder, metacarpal, leg, knee, hip, ankle, metatarsal, phalange, neck, or the like.). It will also be appreciated that the system 10 may be used in connection with any portion of hard tissue portions (e.g. bone, or the like) or soft tissue (e.g., muscle, tendon, ligament, cartilage, or the like) of the native tissue. The system 10 includes a surgical device 12 for use with an operating table. The surgical device 12 maintains an outline or an imprint of a position of the native tissue so that the patient may be returned to that same position for a surgical procedure. The system 10 also may include a PSI 14 that is configured to reflect one or more physical characteristics of the native tissue for use as the patient is positioned on in the surgical device 12.

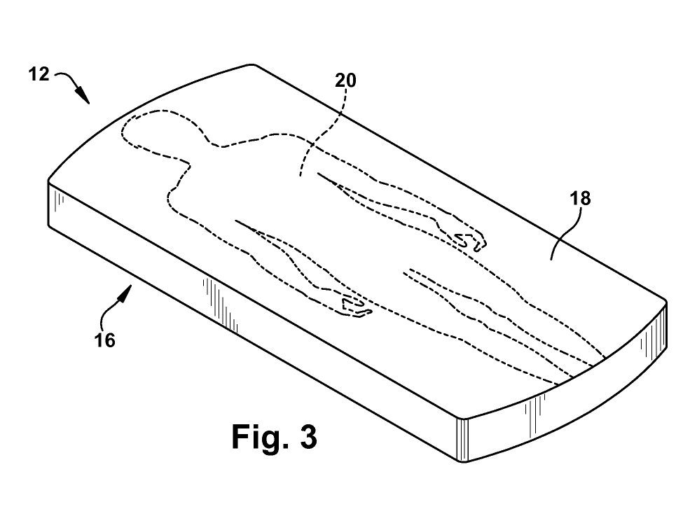

One aspect of the present invention includes a surgical system 10 for positioning, and marking locations on, a patient for a surgical procedure. As shown in FIG. 2, the system 10 may include a surgical device in the form of a rest block 12. The rest block 12 is used for replicating a position of a portion of a contact tissue of a patient on a surgical table for a surgical procedure. As used herein, the term “contact tissue” (and variants thereof) refers to a portion of a patient's body that contacts a portion of the rest block 12. Specifically, the term “contact tissue” is used in the examples described herein to refer to at least a portion of the patient's head, torso, arms, and legs. The rest block 12 is described herein as a singular structure; however, it will be appreciated that the rest block may be integrally formed with another structure (e.g., an operating table). The rest block 12 is manufactured from one or more materials that allow(s) the rest block to accept and permanently maintain an outline or imprint of the contact tissue. In one example, the rest block 12 is substantially made from a polyurethane composition, such as Tempur® material (available from Tempur-Pedic® Management Inc., Lexington, Ky.). In another example, the rest block 12 may be substantially made from a polystyrene composition, such as Styrofoam™ (available from Dow Chemical Co., Midland, Mich.). The rest block 12 may be covered in a thin film of a durable material (e.g., plastic) to prevent unwanted damage to the rest block. As shown in FIG. 2, the rest block 12 is substantially rectangular; however, it will be appreciated that the rest block may have different shapes (e.g., curvilinear, rectilinear, circular, trapezoidal, etc.).

The rest block 12 includes a main body 16 with a footprint-receiving portion 18.

As shown in FIG. 2, the footprint-receiving portion 18 is disposed on a top surface of the main body 16. The footprint-receiving portion 18 is configured to accept and permanently maintain a footprint 20 of at least a portion of the contact tissue in order to assist with later re-positioning of the contact tissue into a position that is substantially the same as the originally-received position. As used herein, the term “footprint” means an imposed deformation on the footprint-receiving portion 18 produced as a result of a portion of the contact tissue contacting the footprint-receiving portion. As used herein, the term “impose” (and variants thereof) refers to the ability of the footprint-receiving portion 18 to reflect, replicate, record, reproduce, mirror, imitate, memorialize, and/or mimic a desired position of at least a portion of a contact tissue for a surgical procedure.

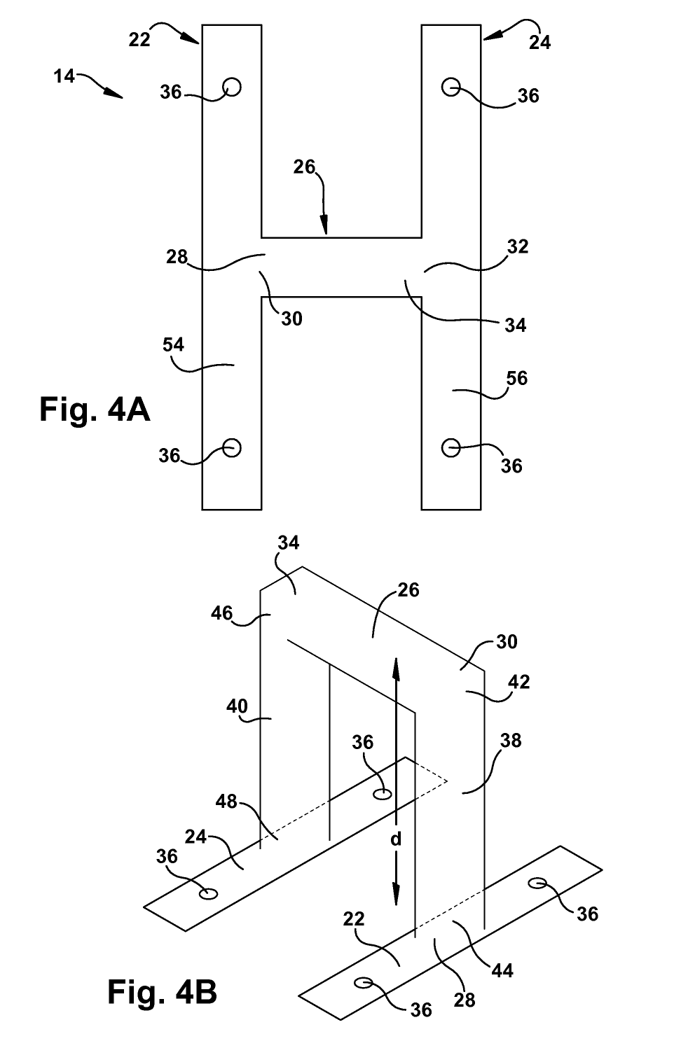

In one instance, the footprint 20 may be a two-dimensional outline embodying a silhouette of the portion of the contact tissue. In another instance, the footprint 20 may be a three-dimensional imprint embodying a contoured profile of the portion of the contact tissue. In a further instance, the footprint 20 may be a combination of the silhouette and the contoured profile of the contact tissue. The footprint 20 is configured to memorialize and impart a previously-established desired position to the native tissue for the surgical procedure. As shown in FIG. 3, the footprint 20 of the portion of the contact tissue may impose a portion of a torso of the patient; however it will be appreciated that a footprint of contact tissue of any portion of the patient's body (e.g., arm, shoulder, metacarpal, leg, knee, hip, ankle, metatarsal, phalange, neck, etc.) may be accepted and permanently maintained by the footprint-receiving portion 18. The footprint 20 is imposed on the footprint-receiving portion 18 when the patient lies on the rest block 12. As the patient lies on the rest block 12, a force produced on the rest block by the weight of the patient causes the footprint-receiving portion 18 to deform and responsively produce the footprint 20 of the native tissue.

When a patient is positioned on the rest block 12, the footprint-receiving portion 18 produces the resulting footprint 20 of position of the contact tissue. The footprint-receiving portion 18 accepts and permanently maintains the position of the patient as the contact tissue contacts the footprint-receiving portion. The footprint 20 is imposed as a result of the applied force of the contact tissue acting on the footprint-receiving portion 18. As shown, a prone position of the patient is imposed on the footprint-receiving portion 18; however, it will be appreciated that the footprint-receiving portion may impose any position of the patient (e.g., a supine position). The footprint 20 is permanently maintained due to the composition of the material of the rest block 12. The rest block 12 helps to impose the position of the contact tissue maintained in the footprint-receiving portion 18 upon return of the same patient to the rest block at a later time. The rest block 12 permanently maintains the footprint 20 so that, at a later time, the patient may be aligned within the position imposed by the footprint. At the time of the surgical procedure, the patient is guided into the position imposed on the footprint-receiving portion 18 in the same position of the contact tissue previously accepted by the footprint-receiving portion.

In another aspect of the present invention, the PSI 14 is used to aid a surgeon in marking locations of the native tissue for a surgical procedure. The PSI 14 is configured to reflect at least one physical characteristic of a portion of the native tissue. In one example, the PSI 14 is configured to reflect at least one physical characteristic of at least a portion of the patient's spine; however, it will be appreciated that the PSI may reflect a portion of another portion of the patient's body (e.g., arm, shoulder, metacarpal, leg, knee, hip, ankle, metatarsal, phalange, neck, etc.). The PSI 14 may be produced from images taken of the native tissue while the patient is positioned on the rest block 12, as described herein.

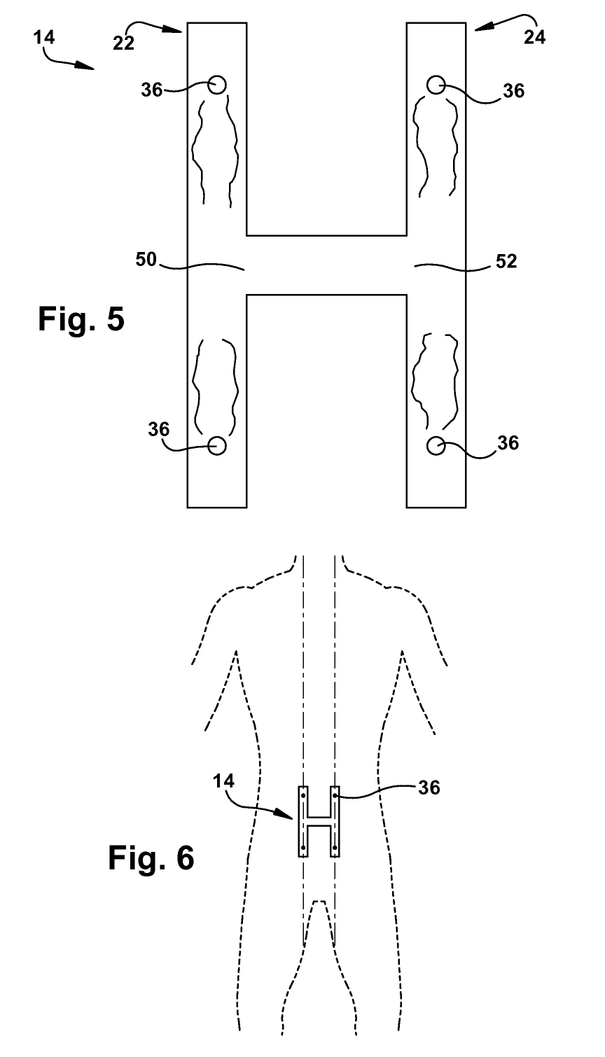

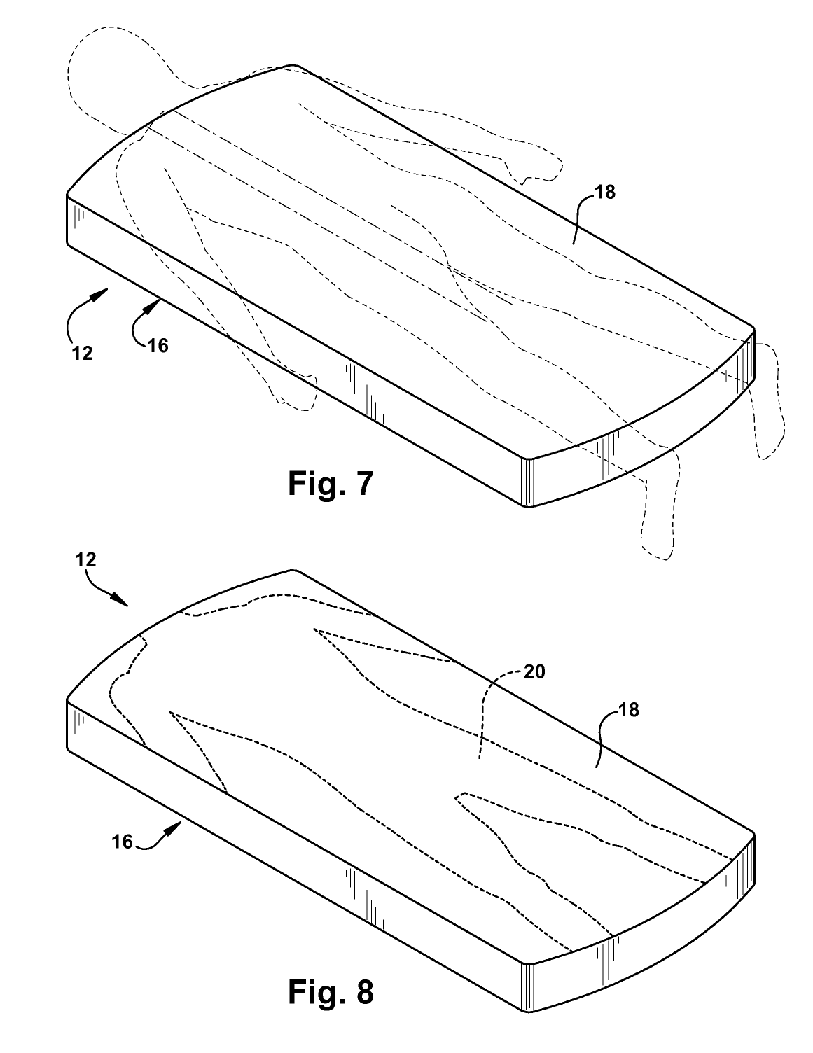

As shown in FIGS. 4A-4B, the PSI 14 may have an H-shaped configuration; however, it will be appreciated that the PSI may have other shapes (e.g., rectangular, circular, triangular, trapezoidal, U-shaped, etc.). The PSI 14 may comprise a plurality of segments; however, it will also be appreciated that the PSI may be formed as a singular integral structure. The PSI 14, as shown in FIGS. 4A-4B, includes a first segment 22 and a second segment 24 joined together by an intermediate segment 26. Each of the first and second segments 22 and 24 may have substantially equal lengths, widths, and thicknesses. The intermediate segment 26 may also have any desired length, width, and thickness. The first segment 22, the second segment 24, and the intermediate segment 26 each have substantially rectangular cross-sectional shape; however it will be appreciated that the first segment, the second segment, and the intermediate segment may have a cross-sectional shape of any desired configuration (e.g., circular, triangular, trapezoidal, etc.).

The first segment 22 includes an intersection area 28 that engages a first end 30 of the intermediate segment 26. The second segment 24 includes an intersection area 32 that engages a second end 34 of the intermediate segment 26. At least one of the first and second segments 22 and 24 includes at least one opening 36 configured to receive a marking structure (not shown) or a fastener (not shown), as described herein, to mark a location on the native tissue for a surgical procedure. The intermediate segment 26 may optionally include at least one opening 36 (not shown). The first segment 22, the second segment 24, and the intermediate segment 26 may each have a generally rectangular-shaped cross-sectional shape; however, it will be appreciated that the cross-sectional shapes of the first segment, the second segment, and the intermediate segment may be other shapes (e.g., square, circular, triangular, and the like).

In another example, as shown in FIG. 4B, the intermediate segment 26 may be connected to the first segment 22 by a first connecting segment 38, and connected to the second segment 24 by a second connecting segment 40 so that the intermediate segment 26 is spaced vertically from the first and second segments by a distance d. A first end 42 of the first connecting segment 38 is connected to the first end 30 of the intermediate segment 26. A second end 44 of the first connecting segment 38 is connected to the intersection area 28 of the first segment 22. Similarly, a first end 46 of the second connecting segment 40 is connected to the second end 34 of the intermediate segment 26. A second end 48 of the second connecting segment 40 is connected to the intersection area 32 of the second segment 24.

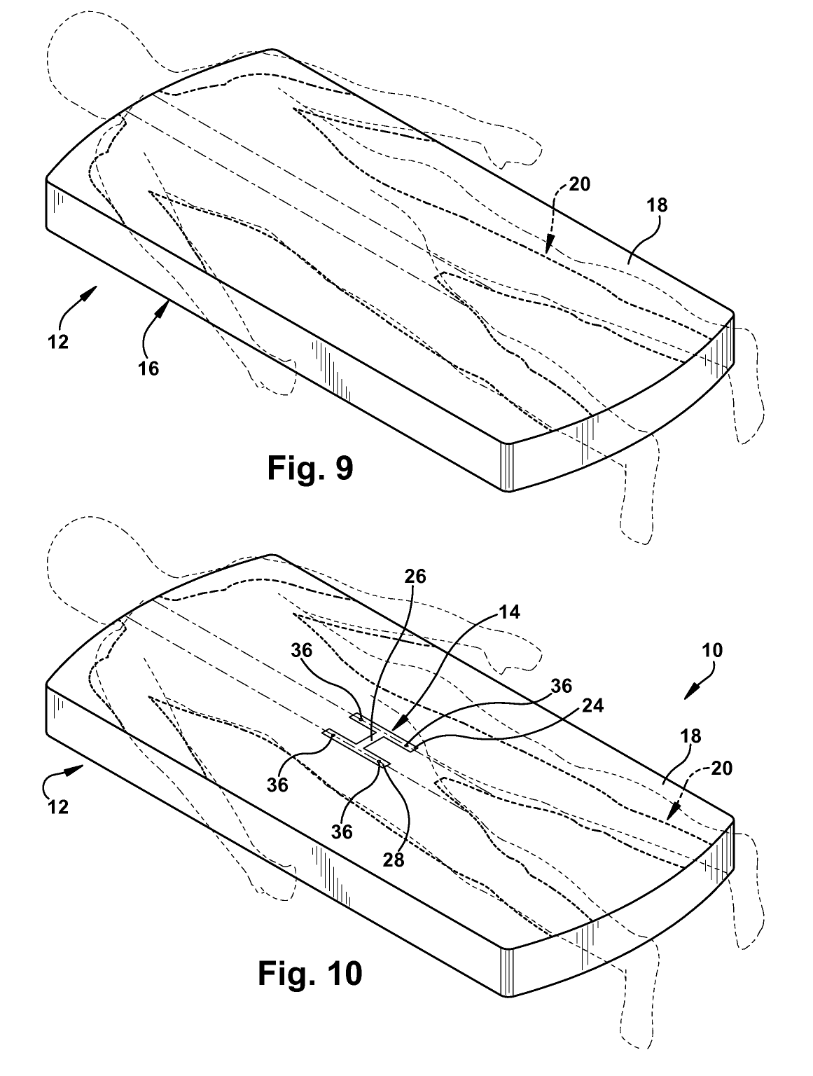

As shown in FIG. 5, the first segment 22 includes a first contact surface 50, and the second segment 24 includes a second contact surface 52. The first and second contact surfaces 50 and 52 each correspond to the shape of the first segment 22 and the second segment 24, respectively. The contact surfaces 50 and 52 may be contoured to mirror one or more physical characteristics of the native tissue. Each of the contact surfaces 50 and 52 are manufactured to correspond to a portion of the subcutaneous tissue (e.g., one or more pedicles of the spine) of which images are taken as the patient lies on the rest block 12, as described herein. The contact surfaces 50 and 52 are configured to mate with the skin surface that overlies the subcutaneous tissue which the contact surfaces correspond to. In one instance, the first contact surface 50 may correspond to a first side of a patient's spine, and the second contact surface 52 may correspond to an opposing second side of a patient's spine. However, it will be appreciated that the first and second contact surfaces 50 and 52 may correspond to one or more sides of another portion of native tissue (e.g., arm, shoulder, metacarpal, leg, knee, hip, ankle, metatarsal, phalange, neck, etc.).

The first and second contact surfaces 50 and 52 are manufactured so that each contact surface substantially matches a corresponding profile of the portion of the subcutaneous tissue. In one instance, each of the contact surfaces 50 and 52 is configured to contact, and mate with the corresponding profile of the portion of the subcutaneous tissue. In another instance, the contact surfaces 50 and 52 are placed on a portion of the skin surface that overlies the portions of subcutaneous tissue. In a further instance, the PSI 14 is positioned such that the contact surfaces 50 and 52 are placed on the skin surface that overlies opposing first and second sides, respectively, of a portion of the patient's spine; however, it will be appreciated that the contact surfaces may instead be placed on the skin surface that overlies one or more sides of another portion of native tissue (e.g., arm, shoulder, metacarpal, leg, knee, hip, ankle, metatarsal, phalange, neck, etc., such as for another type of surgical procedure). It will also be appreciated that the intermediate segment 26 may optionally include a contact surface (not shown), which may correspond to a portion of the patient's body located between the portions of the native tissue that the contact surfaces 50 and 52 correspond to.

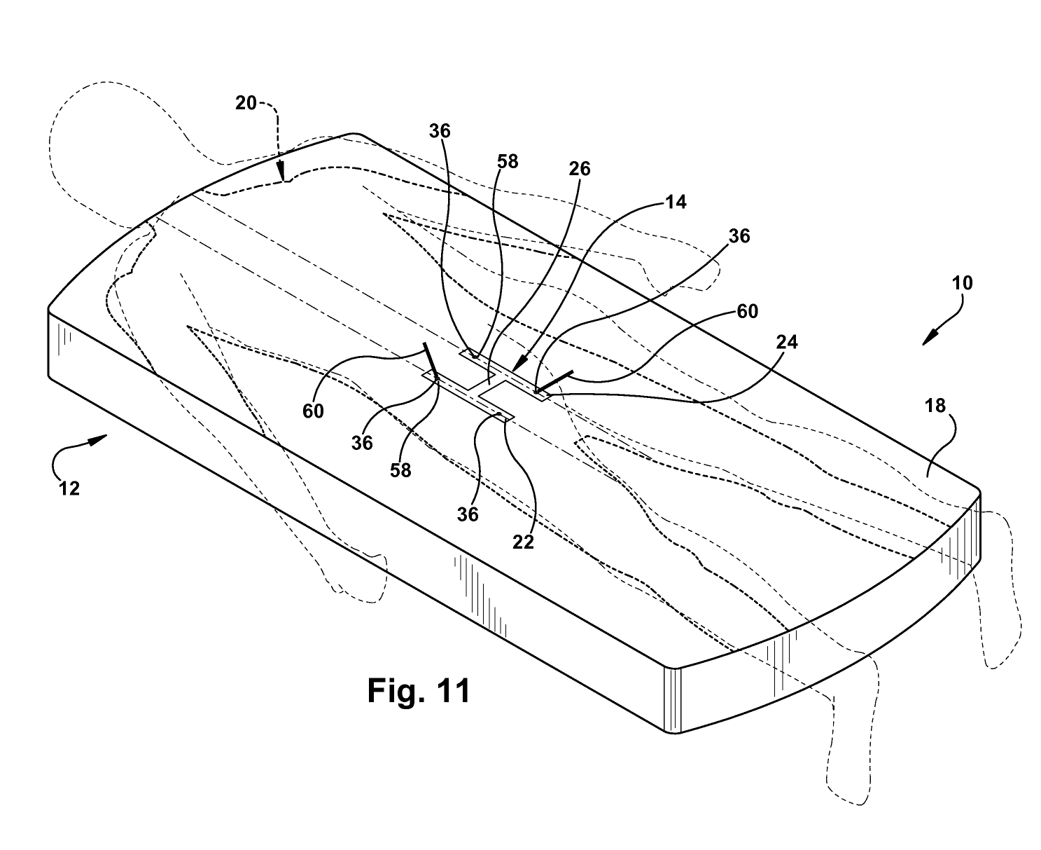

Once the first and second contact surfaces 50 and 52 have been manufactured to correspond to a portion of the subcutaneous tissue, one or more openings 36 are formed into the first and second segments 22 and 24; however, it will be appreciated that the openings may be formed before or during manufacture of the contact surfaces. The openings 36 of the first segment 22 extend from a first top surface 54 (FIG. 4A) of the first segment and through the first contact surface 50 (FIG. 5). Similarly, the openings 36 of the second segment 24 extend from a second top surface 56 (FIG. 4A) of the second segment and through the second contact surface 52 (FIG. 5). As shown in FIG. 6, the PSI 14, with the first and second contact surfaces 50 and 52 formed, is placed on the patient as the patient lies on the rest block 12. Specifically, the PSI 14 mates with the skin surface that overlies the portion of subcutaneous tissue to which the first and second contact surfaces 50 and 52 correspond. Once the first and second contact surfaces 50 and 52 are aligned with the desired portion of the native tissue, the openings 36 are positioned to overlie a specific portion of the subcutaneous tissue. In one instance, the openings 36 each overlie a specific pedicle (not shown) of the patient's spine; however, it will be appreciated that the openings may overlie a specific portion of the spine, or another portion of native tissue (e.g., arm, shoulder, metacarpal, leg, knee, hip, ankle, metatarsal, phalange, neck, etc.). Each opening 36 corresponds to a location and a desired trajectory of a single marking structure (not shown), a single fastener (not shown), or another structure which may be inserted into or through the openings. In one example, the marking structure may be a K-wire, guide pin, or other landmark that is inserted into an opening 36 to mark a location on the subcutaneous tissue. In another example, the fastener may be a bone screw inserted through the opening 36 into the subcutaneous tissue. The fastener is shown in the figures as inserted after the marking structure has been removed from the native tissue; however, it will be appreciated that the fastener is inserted while the marking structure is still installed within the native tissue.

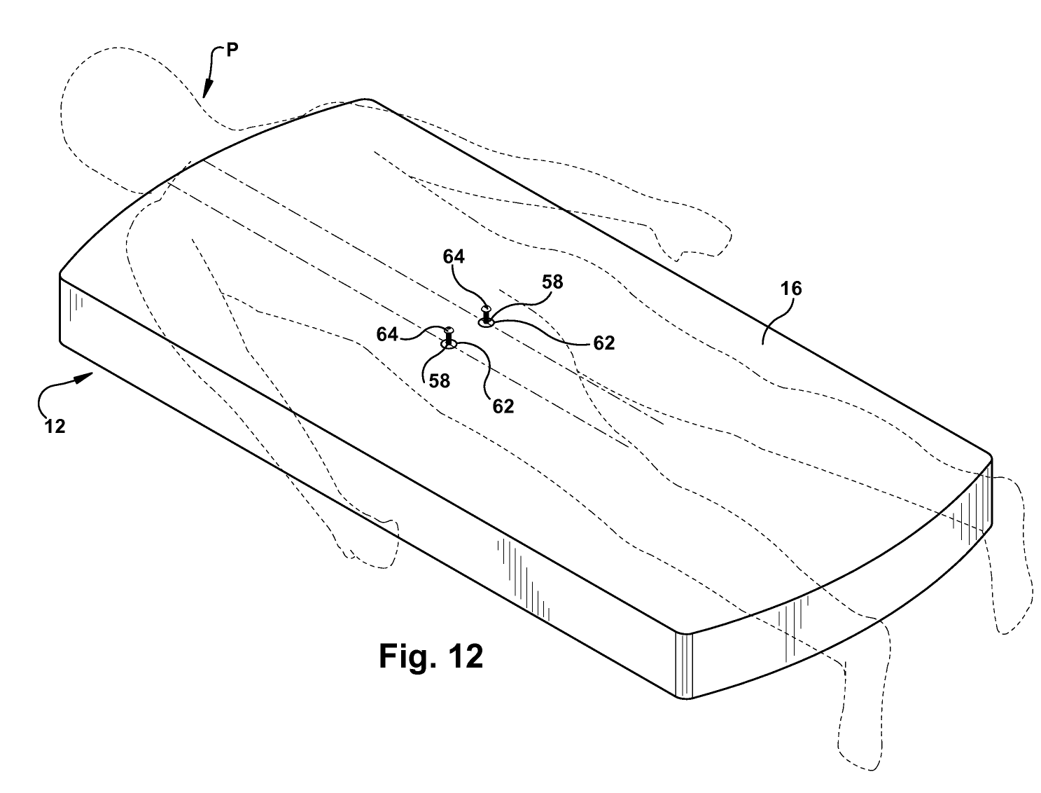

Another aspect of the present invention includes a method for preparing and implementing a preoperative surgical plan. One example of the method is shown in FIGS. 7-11, in which a spinal procedure is illustrated; however, it will be appreciated that the method of the present invention is applicable in other surgical procedures (e.g., arm, shoulder, metacarpal, leg, knee, hip, ankle, metatarsal, phalangeal, neck, and the like), or a relatively non-invasive procedure (e.g., acupuncture, massage, etc.). The described configuration of the system 10, along with other (non-depicted) configurations of the system, may be selected and used by one of ordinary skill in the art to provide a surgical system for positioning and marking locations on different portions of native tissues other than the spine. As with all alternate configurations shown and described herein, description of common elements and operation similar to those in previously described configurations will be omitted, for clarity.

One step of the method may include positioning a patient on the rest block 12 before a spinal procedure to determine a desired position of the patient for the spinal procedure. The desired position of the patient for surgery is imposed in the rest block 12 so that the patient may be returned to that same position imposed in the rest block during the spinal procedure. As shown in FIG. 7, the patient is positioned on the rest block 12 before the spinal procedure so that the desired position (e.g., a prone position) of the patient is replicated on the rest block for the spinal procedure. The footprint-receiving portion 18 accepts the footprint 20 of a portion of the contact tissue that imposes the position of the patient on the rest block for the spinal procedure (FIG. 8).

At the time of the surgical procedure, the patient is aligned within the footprint 20, as shown in FIG. 9, in the same position of the contact tissue replicated by the footprint in the surgical device 12. As shown, the footprint 20 imposes a prone position of the patient; however, it will be appreciated that the footprint may impose another position of the patient (e.g., a supine position). The footprint 20 is used to help guide and align the patient into the position recorded by the rest block 12 at the time of the spinal procedure.

After the patient is aligned on the rest block 12, the PSI 14 is manufactured based on the position of the patient as the patient lies on the rest block. The position needed for the PSI 14 to properly align with the native tissue is substantially the same position of the patient lying on the rest block 12. The PSI 14 may be aligned with a portion of the subcutaneous tissue. In one instance, the PSI 14 may be created to correspond to a profile of a portion of the spine to be operated on during the spinal procedure. Images of the portion of the spine are obtained as the patient lies in the position imposed on the rest block 12. The images capture a profile of the native tissue. In addition, computer aided design software may be used to custom-design the PSI 14 based upon imported data obtained from a computerized tomography scan, or another imaging scan (e.g., digital or analog radiography, magnetic resonance imaging, etc.), of the native tissue. Alternatively, a user may create the PSI 14 without taking images of the spine (e.g., custom-design, software design, etc.).

The PSI 14 is then manufactured responsive to the profile of the subcutaneous tissue captured by the taken, or otherwise obtained, images. In one instance, the PSI 14 may be made from a stock component that may be manipulated to correspond to a portion of the native tissue. The first and second contact surfaces 50 and 52 of the first and second segments 22 and 24 are manufactured responsive to the images so that they correspond to opposing first and second portions, respectively, of the patient's spine; however, it will be appreciated that the contact surfaces may be manufactured so that they correspond to one or more portions of another anatomical element of the patient's body (e.g., arm, shoulder, metacarpal, leg, knee, hip, ankle, metatarsal, phalange, neck, and the like).

The system 10 is then used to position the patient for the surgical procedure. The patient is returned to the same position imposed on the rest block 12, as shown in FIG. 9. When the patient is aligned in the position on the rest block 12, the PSI 14 is aligned with the portion of the spine that the contact surfaces 50 and 52 correspond to. In one example, as shown in FIG. 10, the PSI 14 is placed on the skin surface that overlies the portion of the spine that the contact surfaces 50 and 52 correspond to. Once the PSI 14 is positioned over a portion of the spine, the openings 36 are positioned to overlie a specific portion 58 of the subcutaneous tissue. In one instance, when the PSI 14 overlies a portion of the patient's spine, the openings 36 overlie a specific pedicle 62 of the patient's spine.

Once the PSI 14 is positioned on the patient, the PSI is used to determine locations on the native tissue where a surgical marker may be placed. As shown in FIG. 11, at least one surgical marker 60 is inserted through the openings 36 and into one of the surgical apertures so that the surgical marker is positioned adjacent or into the specific pedicle 58 of the spine. The surgical marker 60 corresponds to the position and trajectory of one of the openings 36. In one instance, the surgical marker 60 may be a K-wire or other surgical pin or relatively stiff wire. The K-wires mark locations 62 within or adjacent to the spine. In one example, the locations 62 may be located on or adjacent one or more pedicles of the patient's spine.

As shown in FIG. 12, at least one surgical fastener 64 is then inserted into the spine. The surgical fastener 64 is inserted into the spine at the locations 62 marked by the surgical markers 60. In one instance, the surgical fastener 64 may be a bone screw. The surgical fastener 64 is inserted into the spine at each location 62 (e.g., the pedicles) that is marked by the surgical markers 60 during or after the spinal procedure in which the PSI 14 is used.

While aspects of the present invention have been particularly shown and described with reference to the preferred embodiment above, it will be understood by those of ordinary skill in the art that various additional embodiments may be contemplated without departing from the spirit and scope of the present invention. For example, the specific methods described above for using the described system are merely illustrative; one of ordinary skill in the art could readily determine any number of tools, sequences of steps, or other means/options for virtually or actually placing the above-described apparatus, or components thereof, into positions substantially similar to those shown and described herein. Any of the described structures and components could be integrally formed as a single piece or made up of separate sub-components, with either of these formations involving any suitable stock or bespoke components and/or any suitable material or combinations of materials; however, the chosen material(s) should be biocompatible for most applications of the present invention. The mating relationships formed between the described structures need not keep the entirety of each of the “mating” surfaces in direct contact with each other but could include spacers or holdaways for partial direct contact, a liner or other intermediate member for indirect contact, or could even be approximated with intervening space remaining therebetween and no contact. Though certain components described herein are shown as having specific geometric shapes, all structures of the present invention may have any suitable shapes, sizes, configurations, relative relationships, cross-sectional areas, or any other physical characteristics as desirable for a particular application of the present invention. Any structures or features described with reference to one embodiment or configuration of the present invention could be provided, singly or in combination with other structures or features, to any other embodiment or configuration, as it would be impractical to describe each of the embodiments and configurations discussed herein as having all of the options discussed with respect to all of the other embodiments and configurations. The system is described herein as being used to plan and/or simulate a surgical procedure of implanting one or more prosthetic structures into a patient's body, but also or instead could be used to plan and/or simulate any surgical procedure, regardless of whether a non-native component is left in the patient's body after the procedure. A device or method incorporating any of these features should be understood to fall under the scope of the present invention as determined based upon the claims below and any equivalents thereof.

Other aspects, objects, and advantages of the present invention may be obtained from a study of the drawings, the disclosure, and the appended claims.

Having described the invention, we claim:

1. A device for positioning a patient on a surgical table for a surgical procedure, the device comprising:
a main body with a footprint-receiving portion configured to accept and permanently maintain a footprint of at least a portion of a contact tissue of a patient's body, the footprint imposing a desired position of the contact tissue for the surgical procedure.

2. The device of claim 1, wherein the footprint-receiving portion is configured to accept a position of the contact tissue when the portion of contact tissue contacts at least a portion of the main body.

3. The device of claim 2, wherein the contact tissue is positioned on the footprint-receiving portion of the main body in substantially the same position imposed on the footprint-receiving portion after the footprint is accepted and permanently maintained.

4. The device of claim 1, wherein the device is substantially made from polyurethane foam.

5. The device of claim 1, wherein the footprint is a two-dimensional outline embodying a silhouette of the contact tissue.

6. The device of claim 1, wherein the footprint is a three-dimensional imprint embodying a contoured profile of the contact tissue.

7. An system for positioning a patient on a surgical table for a surgical procedure and implementing a preoperative surgical plan, the system comprising:
a surgical device having a main body with a footprint-receiving portion configured to accept and permanently maintain a footprint of at least a portion of a contact tissue, the footprint imposing a desired position of the contact tissue for a surgical procedure; and
a patient-specific instrument configured to reflect characteristics of at least a portion of a native tissue, the patient-specific instrument being manufactured responsive to images taken of the portion of native tissue while the patient is positioned on the surgical device.

8. The system of claim 7, wherein the footprint-receiving portion is configured to accept a position of the contact tissue when the contact tissue contacts at least a portion of the main body.

9. The system of claim 8, wherein the contact tissue is positioned on the footprint-receiving portion of the main body in substantially the same position imposed on the footprint-receiving portion.

10. The system of claim 7, wherein the surgical device is substantially made from polyurethane foam.

11. The system of claim 7, wherein the footprint is a two-dimensional outline embodying a silhouette of the contact tissue.

12. The system of claim 7, wherein the footprint is a three-dimensional imprint embodying a contoured profile of the contact tissue.

13. The system of claim 7, wherein the patient-specific instrument includes at least one feature that corresponds to the native tissue of which the images were taken.

14. The system of claim 7, wherein the patient-specific instrument is placed on a portion of a skin surface overlying the corresponding portion of the native tissue.

15. The system of claim 7, wherein the patient-specific instrument includes at least one opening, each opening corresponding to a location and a desired trajectory of a fastener to be inserted into the native tissue.

16. The system of claim 15, wherein at least one marking structure is inserted into the native tissue through the opening.

17. The system of claim 16, wherein at least one fastener is inserted through the opening at a location in the native tissue that corresponds to a location where the at least one marking structure is inserted.

18. A method for preparing and implementing a preoperative surgical plan, the method comprising the steps of:
positioning a patient on a surgical device before a surgical procedure, the surgical device having a footprint-receiving portion configured to accept and permanently maintain a footprint of at least a portion of a contact tissue as arranged in a desired position for the surgical procedure, the footprint-receiving portion being configured to impose a position of the contact tissue when the contact tissue contacts at least a portion of the main body;
returning the patient to the position imposed in the footprint-receiving portion of the main body;
aligning a patient-specific instrument with at least a portion of a native tissue;
forming at least one surgical aperture into the native tissue guided by at least one opening of the patient-specific instrument;
and marking at least one location within the at least one surgical aperture with a marking structure.

19. The method of claim 18, wherein the surgical device is substantially made from polyurethane foam.

20. The method of claim 18, wherein the footprint is a two-dimensional outline embodying a silhouette of the contact tissue.

21. The method of claim 18, wherein the footprint is a three-dimensional imprint embodying a contoured profile of the contact tissue.

22. The method of claim 18, further including the step of creating the patient-specific instrument, the patient-specific instrument being configured to correspond to the native tissue, including the steps of:
obtaining images of the native tissue of the patient while the patient is positioned on the surgical device in the desired position before the surgical procedure;
and manufacturing the patient-specific instrument responsive to the images such that the patient-specific instrument includes at least a portion that corresponds to the native tissue of which the images were taken.

23. The method of claim 18, wherein the step of aligning a patient-specific instrument with the native tissue of the patient includes the step of placing the patient-specific instrument on a skin surface overlying the corresponding native tissue.

24. The method of claim 18, wherein the step of marking locations within the at least one surgical aperture for a surgical fastener includes the step of inserting at least one marking structure into the native tissue through the opening.

25. The method of claim 18, further including the step of inserting at least one surgical fastener through the opening into the native tissue at each location where the marking structures are inserted.
